(19) United States
(12) Patent Application Publication
(10) Pub. No.: US 2020/0316224 A1
(43) Pub. Date: Oct. 8, 2020

(54) Method and Kit for Treatment of Genetic Congenital Birth Defects

(71) Applicants: Elise Kendall Patchett, Woodbury, MN (US); Kiley Marie Robertson, Woodbury, MN (US)

(72) Inventors: Elise Kendall Patchett, Woodbury, MN (US); Kiley Marie Robertson, Woodbury, MN (US)

(21) Appl. No.: 16/839,317

(22) Filed: Apr. 3, 2020

Related U.S. Application Data
(60) Provisional application No. 62/919,941, filed on Apr. 5, 2019.

(51) Int. Cl. A61K 48/00

(52) U.S. Cl. CPC A61K 48/0066; A61K 48/0083; A61K 48/0075

(57) ABSTRACT

A method of treating a congenital birth defect includes detecting the presence of at least one mutated gene associated with a birth defect and injecting foreign genetic material containing at least one non-mutated version of the detected mutated gene into a patient, thereby promoting a desired therapeutic outcome in the patient. A kit for treating a congenital birth defect in a patient comprising normal genetic material tailored to treat the congenital birth defect and an injection device for providing the normal genetic material to the patient is also described.

### CROSS REFERENCE TO RELATED APPLICATION

This application claims the benefit of Provisional Patent Application No. 62/919,941, filed Apr. 5, 2019, the disclosure of which is incorporated by reference herein in its entirely.

### BACKGROUND

The present invention relates generally to medical treatments and, more particularly, to the treatment of genetic congenital birth defects.

Gene mutations may cause or contribute to certain birth defects such as, for example, congenital heart defects. A birth defect is a health condition that is present in a baby at birth. Non-genetic influences (i.e. environmental factors), such as smoking, drinking alcohol or exposure to harmful chemicals, may also contribute to the development of birth defects. Genetic screening is available to determine if a baby is at risk for certain genetic conditions. If a screening test shows that a baby may be at risk for a certain condition, diagnostic tests, such as amniocentesis or chorionic villus sampling, are available to learn more about the baby's condition and likelihood of developing a genetic condition.

Congenital heart defects include, for example, hypoplastic left heart syndrome (HLHS), pulmonary atresia, Tetralogy of Fallot, total anomalous pulmonary venous return, transposition of the great arteries, tricuspid atresia and truncus arteriosus. Hypoplastic left heart syndrome (HLHS) is a congenital heart condition that arises during gestation in which the left ventricle is underdeveloped or nonexistent, preventing the heart from properly circulating blood throughout the body. Although the heart has significant abnormalities, blood can circulate to the body through the aorta with only the right ventricle working. An opening between the left and right ventricles, known as the foramen ovale, typically closes within a few days after birth. However, in infants with HLHS, the foramen ovale must remain open in order for blood to circulate properly through the body. Thus, medication may be administered to stabilize the opening until surgery can be performed.

The cause of HLHS is not fully understood, but some cases seem to have genetic components. Environmental factors also seem to play a role. Without life-prolonging intervention, HLHS is fatal. 95% of untreated infants with HLHS die in the first weeks of life.

Surgical operations may be used to treat patients diagnosed with HLHS and may extend life into adulthood. Such surgery, however, is complex and risky. In addition, patients who successfully undergo surgery require monitoring by a cardiologist for the rest of their lives to check on their heart function. Many also experience motor, cognitive or language impairments.

Individuals with HLHS who undergo surgery typically undergo several surgeries to improve heart function. Soon after a baby is born, the baby undergoes a first surgery in order to increase blood flow by rerouting blood away from the underdeveloped left ventricle. By altering the course of blood flow, the right ventricle becomes the main pumping chamber of the heart. Surgeries do not cure HLHS, but help to ensure the body stays properly oxygenated. Many individuals must still undergo a heart transplant later in life due to overworking of the right ventricle.

Mechanical devices, such as implants or shunts, are known in the patented art to help improve blood flow. U.S. Pat. No. 6,468,303 (Amplatz et. al.), for example, describes a collapsible medical device and associated method for shunting selected organs and vessels wherein the medical device is shaped from a shape memory metal fabric.

### SUMMARY

There is a need for improved methods of treating genetic congenital birth defects. In particular, it would be desirable to provide an easier, less invasive, (e.g. non-surgical), and more effective treatment option for genetic congenital birth defects.

In one aspect, the present disclosure provides a non-surgical method of treating congenital birth defects having a genetic component. In one aspect, having a genetic component means one or more genes appear to be associated with or appear to be contribute the likelihood that a particular individual will develop a particular congenital birth defect. In one embodiment, the present disclosure provides a method of treating a congenital heart defect having a genetic component.

In one embodiment, the present disclosure provides a method of treating a congenital birth defect comprising the steps of detecting the presence of at least one mutated gene associated with a congenital birth defect, and providing foreign genetic material containing at least one normal (i.e. non-mutated) version of the detected gene mutation to a patient, thereby promoting a desired therapeutic outcome in the patient.

In another embodiment, the present disclosure provides a method of treating a congenital birth defect comprising the steps of identifying at least one individual having an increased risk of developing a congenital birth defect and providing genetic material containing at least one non-mutated version of a gene mutation detected in the individual that is associated with the congenital birth defect, thereby promoting a desired therapeutic outcome in the patient. In a specific embodiment the individual is a fetus.

In one aspect, the congenital birth defect may be a congenital heart defect. In a more specific aspect, the congenital heart defect may be hypoplastic left heart syndrome (HLHS).

In other aspects and embodiments, the step of providing foreign genetic material may be done via gene therapy, the foreign genetic material may be at least one of DNA and RNA, and/or the gene therapy may involve injecting at least one of a viral vector, non-viral and synthetic (e.g. chemical) vector into the patient. In other aspects and embodiments, the detected gene mutation may be at least one of the NKX2.5 and HAND1 genes, the detected gene mutation may include mutations within both the NKX2.5 and HAND1 genes, the step of detecting the presence of at least one mutated gene may comprise genetic testing in combination with an evaluation or assessment of an individual's exposure to air pollution during gestation, the foreign DNA may be delivered as part of a viral vector, the virus may be at least one of a retrovirus, adenovirus, adeno-associated virus and herpes simplex virus, the adenovirus may be injected into a placenta, the injection may be administered via chorionic villus, and/or the injection may be administered during the first trimester of pregnancy.

In another aspect, the present disclosure provides a kit for treating a congenital birth defect. In one embodiment, the kit may include normal genetic material tailored to treat the congenital birth defect, and an injection device for providing the normal genetic material to a patient. The kit may also include an extraction device for removing genetic material (e.g. amniotic fluid) from the patient. In more specific embodiments, the extraction device may include a first hollow sterile needle, the injection device may include a second hollow sterile needle, the normal genetic material may include at least one non-mutated version of a gene mutation detected in the patient, the foreign genetic material may be injected into the placenta of the patient, and/or the normal genetic material may include an adenovirus.

Even though genetic testing may not be currently available to detect a specific gene mutation associated with a particular congenital birth defect or health condition, genetic testing capability is advancing rapidly. Accordingly, the present invention contemplates the treatment of genetic congenital birth defects that cannot be detected using known techniques but may be detectable in the future based on advances in genetic testing.

As used herein, the term “patient” may refer to a pregnant individual, an infant or a fetus.

Advantages of certain embodiments of the invention described herein include providing a treatment for congenital birth defects that is less invasive and corrects the congenital birth defect in-utero, thereby allowing the fetus to develop normally.

### DETAILED DESCRIPTION

The present disclosure relates to methods and kits for the treatment of congenital birth defects believed to have a genetic component, such as, for example, congenital heart defects such as hypoplastic left heart syndrome (HLHS), pulmonary atresia, Tetralogy of Fallot, total anomalous pulmonary venous return, transposition of the great arteries, tricuspid atresia, truncus arteriosus, patent ductus arteriosus (PDA), atrial septal defect, ventricular septal defect and Tetralogy of Fallot.

It will be recognized that the methods and kits described herein may also be used for the treatment of non-heart related congenital birth defects believed to have a genetic component, such as, congenital anomalies of the nervous system including, for example, spina bifida, Arnold-Chiari malformation, cleft lip and cleft palate, club foot, sickle cell disease, cystic fibrosis, Tay-Sachs disease, hemophilia, Marfan syndrome, Down syndrome, Patau syndrome (e.g. trisomy 13 or trisomy D), and Edwards syndrome (i.e. trisomy 18). Accordingly, the present disclosure is not limited to the treatment of a particular congenital birth defect so long as the congenital birth defect can be detected via genetic testing (e.g. genetic screening or diagnostic testing), and the treatment of the detected congenital birth can be accomplished by providing to a patient (i.e. a pregnant mother or fetus) foreign genetic material containing at least one non-mutated version of the detected gene mutation. Genetic testing may include, for example, genetic screening as well as diagnostic tests and technologies such as chorionic villus sampling (CVS), amniocentesis, fetal blood sampling or percutaneous umbilical blood sampling (PUBS), prenatal chromosome analysis (karyotype), prenatal chromosomal microarray analysis (CMA), and fetal genomic or whole-exome sequencing (WES).

Accordingly, a method of treating a genetic congenital birth defect comprises the steps of detecting the presence of at least one mutated gene associated with a congenital birth defect, and providing foreign genetic material containing at least one non-mutated version of the detected gene mutation to a patient, thereby promoting a desired therapeutic outcome in the patient. In a more specific embodiment, the detected congenital birth defect is a congenital heart defect.

In one aspect, the step of providing foreign genetic material is done via gene therapy or gene transfer. The foreign genetic material may include at least one of DNA and RNA. In one embodiment, gene therapy may involve injecting at least one of a viral vector, non-viral and synthetic (e.g. chemical) into the patient. Suitable viruses may include retroviruses, adenoviruses, herpes simplex, vaccinia and adeno-associated virus. Methods for non-viral gene therapy may include the injection of naked DNA, electroporation, sonoporation, magnetofection, and the use of oligonucleotides and inorganic nanoparticles. In a specific embodiment, foreign DNA is delivered to a patient as part of a viral vector, and the viral vector may comprise, for example, at least one of a retrovirus, lentivirus, adenovirus, adeno-associated virus and herpes simplex virus.

In a specific aspect, the viral vector may comprise an adenovirus injected into a placenta of a patient. Injection into the placenta of a patient may minimize or reduce the likelihood of complications or undesirable side effects caused by the treatment. In a more specific embodiment, the injection is administered during the first trimester of pregnancy. Administering foreign genetic material containing at least one non-mutated version of the detected gene mutation to a patient during the first trimester may increase the efficacy of the treatment because it allows the non-mutated version of the gene more time to correct the gene mutation (i.e. because it occurs earlier in the gestational period of the fetus).

In a specific embodiment, the congenital heart defect is hypoplastic left heart syndrome (HLHS). In this embodiment, the method includes detecting a gene mutation in at least one of the NKX2.5 and HAND1 genes. While not wishing to be limited by theory, it is believed that mutations within the NKX2.5 and HAND1 genes, possibly in combination with exposure to air pollution during gestation, may result in abnormal development of the left ventricle, ultimately causing HLHS. NKX2.5 is a transcriptional factor that is responsible for regulation of DNA transcription during development of the embryonic heart. This factor is important for normal heart formation and development. Researchers have studied the link between NKX2.5 and HLHS, and many have found that individuals with HLHS have a mutation within the NKX2.5 gene that results from either a nucleotide substitution, frame deletion, or premature termination of DNA transcription. Mutations of the NKX2.5 gene can be spread throughout the coding region of the gene. However, not all HLHS patients with an NKX2.5 mutation exhibit the same NKX2.5 mutation signifying that multiple different mutations of NKX2.5 may result in the same phenotype.

In mice, mutation of NKX2.5 has been seen to cause patient foramen ovale and atrial septal defect. A different study that looked at NKX2.5 gene mutations among patients with congenital heart disease found that 13 patients with conotruncal heart malformations had mutations within the NKX2.5 factor. These results suggest the mutation of NKX2.5 causes heart malformations commonly seen among patients with HLHS, thus exhibiting a possible causal relationship between NKX2.5 mutation and occurrence of HLHS. However, despite the relationship between NKX2.5 mutation and HLHS, not everyone diagnosed with HLHS has a mutation with their NKX2.5 gene. This suggests the possibility that multiple factors are involved in the development of HLHS.

In addition to mutations within the NKX2.5 gene, there is significant evidence that suggests mutations in the HAND1 gene simultaneously play a role in the cause of HLHS. The HAND1 gene belongs to the Class B (tissue-specific) basic helix-loop-helix (bHLH) group of transcription factors, giving it a crucial role in placentation and cardiac morphogenesis, dorso-ventral patterning, and interventricular septum formation in the embryonic heart. Therefore, should a mutation occur on the HAND1 gene, the developing embryonic heart lacks proper “instructions” for development, which may result in heart defects such as HLHS. A study that sequenced the human HAND1 gene in heart tissues derived from 31 unrelated patients diagnosed with hypoplastic hearts found that in 24 of 31 hypoplastic ventricles, a common frameshift mutation in the bHLH domain was detected which impaired HAND1 function. Due to the absence of this specific mutation in all of the hypoplastic ventricles, at least one environmental factor, in addition to genetic mutations, likely contributes to the development of HLHS.

Air pollution, which refers to the presence of particulate matter in the atmosphere, also appears to be a factor in the cause of HLHS for a variety of reasons, specifically due to its presence in nearly every part of the world in some form. Particulate matter (PM) is a mixture of particles suspended in air that are the result of, for example, motor vehicle emissions, power generation and industrial combustion, fires, construction and agriculture. Particulate matter often contains chemicals that can be harmful to humans, and in some cases, are related to birth defects. A study performed on pregnant women in Hohhot, China revealed that significant associations were found between exposures to CO and PM2.5 and cancer risk, suggesting that air pollutants produced during the heating season, during which coal combustion is used and produces relatively high amounts of air pollution, impacts the presence of HLHS. An additional study in California evaluated the chances of 27 congenital heart defects with respect to quartiles of seven ambient air pollutant and traffic exposures during the first 2 months of pregnancy, as the earlier stages in pregnancy tend to be the most sensitive. This study revealed that PM10 was associated with pulmonary valve stenosis and perimembranous ventricular septal defects, making PM10 and high traffic density likely contributors to heart defects including HLHS.

Thus, in one embodiment of the present disclosure, to counteract the harmful effects that genetic mutations have on fetal cardiac development, placental injection therapy is used for fetuses in which early genetic testing reveals mutated HAND1 and/or NKX2.5 genes. The particular gene delivery technique is not critical to the invention hereof, as long as it achieves the desired outcome. Gene therapy may be accomplished, for example, by inserting DNA into cells through the use of viral vectors in order to restore the proper function of a mutated gene. In a specific embodiment, the virus used as a vector for the therapy is an adenovirus. Adenovirus has demonstrated reasonable efficiency in previous gene therapy trials, and has the advantage of infecting nondividing cells. DNA from donors with genes tested for proper cardiac development are extracted and infused into the adenovirus and made into an injection. When the injection is administered during, for example, the first trimester, the healthy DNA in the adenovirus enters the bloodstream and proper coding for the heart will be accessible, thus preventing the formation of a hypoplastic heart.

In one embodiment, the adenovirus carrying the normal gene is received by the fetus via placental injection. In a more specific embodiment, the desired immune tolerance may be induced through fetal injection, and an ultrasound may be used for identification of the placenta and the proper injection site. The DNA may be injected into the placenta so the DNA can enter the bloodstream. In an alternate embodiment, the process may include injecting normal HAND1 and/or NKX2.5 gene into a hypoplastic heart to ensure proper embryonic heart formation.

In another embodiment, the step of detecting the presence of at least one mutated gene comprises genetic testing in combination with evaluation of at least one environmental factor associated with the congenital birth defect. For example, the evaluation of environmental factors may include an assessment of exposure to air pollution during gestation. That is, genetic testing may be combined with an evaluation of an individual's exposure to environmental factors associated with congenital birth defects which may include, for example, exposure to air pollution during gestation. An individual's exposure to environmental factors may be assessed by, for example, gathering information via an interview or using questionnaires, reviewing air quality data over a time period of interest and/or within a geographic area of interest, evaluating lifestyle factors and/or biologic testing.

In another embodiment, the method for treatment of a congenital birth defect comprises the steps of identifying individuals having an increased risk of developing a congenital birth defect and providing genetic material containing at least one non-mutated version of a gene mutation detected in the individual associated with the congenital birth defect, thereby promoting a desired therapeutic outcome in the patient. In this embodiment, the individual is a fetus.

In another aspect, a kit for treating a congenital birth defect in a patient is provided. The kit may include normal or un-mutated genetic material tailored to treat the congenital birth defect and an injection device for providing the normal genetic material to the patient (e.g. a pregnant mother or a fetus). The injection device may include a sterile needle, the normal genetic material may include at least one non-mutated version of a gene mutation detected in the patient, and the foreign genetic material may be injected into the placenta of the patient. In a specific embodiment, the normal genetic material may be administered via an adenovirus.

Persons of ordinary skill in the art may appreciate that various changes and modifications may be made to the invention described above without deviating from the inventive concept. For example, while the present disclosure describes a method of treating HLHS, it will be recognized that the method described herein may be used to treat other genetic congenital birth defects including, for example, other congenital heart defects such as patent ductus arteriosus, atrial septal defect, ventricular septal defect and Tetralogy of Fallot, congenital anomalies of the nervous system, such as spina bifida, Arnold-Chiari malformation, and cleft palate. Thus, the scope of the present invention should not be limited to the specific methods described in this application, but only by the methods described by the language of the claims and the equivalents of those methods.

What is claimed is:

1. A method of treating a congenital birth defect, comprising the steps of:
(a) detecting the presence of at least one mutated gene associated with a congenital birth defect; and
(b) providing foreign genetic material containing at least one non-mutated version of the detected gene mutation to a patient, thereby promoting a desired therapeutic outcome in the patient.

2. A method of treating a congenital birth defect as defined in claim 1, wherein the congenital birth defect is a congenital heart defect.

3. A method of treating a congenital birth defect as defined in claim 2, wherein the step of providing foreign genetic material is done via gene therapy.

4. A method of treating a congenital birth defect as defined in claim 3, wherein the foreign genetic material is at least one of DNA and RNA.

5. A method of treating a congenital birth defect as defined in claim 4, wherein the gene therapy involves injecting at least one of a viral vector, non-viral and synthetic into the patient.

6. A method of treating a congenital birth defect as defined in claim 5, wherein the congenital heart defect is hypoplastic left heart syndrome (HLHS).

7. A method of treating a congenital birth defect as defined in claim 6, wherein the detected gene mutation is at least one of the NKX2.5 and HAND1 genes.

8. A method of treating a congenital birth defect as defined in claim 6, wherein the detected gene mutation includes mutations within the NKX2.5 and HAND1 genes.

9. A method of treating a congenital birth defect as defined in claim 1, wherein the step of detecting the presence of at least one mutated gene comprises genetic testing in combination with evaluation of at least one environmental factor associated with the congenital birth defect.

10. A method of treating a congenital birth defect as defined in claim 9, wherein the at least one environmental factor includes exposure to air pollution during gestation.

11. A method of treating a congenital birth defect as defined in claim 4, wherein the foreign DNA is delivered as part of a viral vector.

12. A method of treating a congenital birth defect as defined in claim 11, wherein the virus is at least one of a retrovirus, adenovirus, adeno-associated virus and herpes simplex virus.

13. A method of treating a congenital birth defect as defined in claim 12, wherein the adenovirus is injected into a placenta.

14. A method of treating a congenital birth defect as defined in claim 13, wherein the injection is administered via chorionic villus.

15. A method of treating a congenital birth defect as defined in claim 14, wherein the injection is administered during the first trimester of pregnancy.

16. A method of treating a congenital birth defect, comprising the steps of:
(a) identifying individuals having an increased risk of developing a congenital birth defect having a genetic component; and
(b) providing genetic material containing at least one non-mutated version of a gene mutation detected in the individual associated with the congenital birth defect, thereby promoting a desired therapeutic outcome in the patient.

17. A method of treating a congenital birth defect as defined in claim 16, wherein the individual is a fetus.

18. A kit for treating a congenital birth defect in a patient, comprising:
(a) normal genetic material tailored to treat the congenital birth defect; and
(b) an injection device for providing the normal genetic material to the patient.

19. A kit for treating a congenital birth defect as defined in claim 18, wherein the injection device includes a sterile needle, the normal genetic material includes at least one non-mutated version of a gene mutation detected in the patient, and the foreign genetic material is injected into the placenta of the patient.

20. A kit for treating a congenital birth defect as defined in claim 19, wherein the normal genetic material includes an adenovirus.
